(19) United States
(12) Patent Application Publication
(10) Pub. No.: US 2008/0125451 A1
(43) Pub. Date: May 29, 2008

(54) VINORELBINE DERIVATIVES

(75) Inventors: Mark A. WOLF, Delanson, NY (US); Peter R. GUZZO, Niskayuna, NY (US); Ian L. SCOTT, Monroe, WA (US)

Correspondence Address: NIXON PEABODY LLP - PATENT GROUP, 1100 CLINTON SQUARE, ROCHESTER, NY, 14604, US

(73) Assignee: AMR TECHNOLOGY, INC., Manchester Center, VT (US)

(21) Appl. No.: 11/854,316

(22) Filed: Sep. 12, 2007

Related U.S. Application Data
(60) Provisional application No. 60/843,940, filed on Sep. 12, 2006.

(51) Int. Cl. A61K 31/437; C07D 471/16; A61P 35/00

(52) U.S. Cl. 514/283; 546/51

(57) ABSTRACT

The present invention relates to novel vinorelbine derivatives. Pharmaceutical compositions containing these compounds as well as processes of preparation and processes of use for treatment of various conditions are also disclosed.

The present invention claims benefit of U.S. Provisional Application Ser. No. 60/843,940, filed Sep. 12, 2006, which is hereby incorporated by reference in its entirety.

### FIELD OF THE INVENTION

The present invention relates to vinorelbine derivatives which are potent inhibitors of cellular mitosis and proliferation, as well as pharmaceutical compositions, preparation processes, and methods of use for treatment of various conditions.

### BACKGROUND OF THE INVENTION

### Cellular Proliferation and Cancer

The disruption of external or internal regulation of cellular growth can lead to uncontrolled cellular proliferation and in cancer, tumor formation. This loss of cellular growth control can occur at many levels and, indeed, does occur at multiple levels in most tumors. Under these circumstances, although tumor cells can no longer control their own proliferation, such cells still must use the same basic cellular machinery employed by normal cells to drive their growth and replication.

### Mitosis and Spindle Formation

In a process known as mitosis, cancer cells, like all mammalian cells, multiply through replication and segregation of the original chromosomes. Following DNA replication in the S phase, the cells progress in the G2 phase. During the G2 phase, cells continue to increase in mass and prepare for mitosis. If chromosome damage is present in the G2 phase, the affected cell responds by activating the G2 phase checkpoint, which prevents progression into mitosis. In the absence of DNA damage or following repair of damage, the G2 phase cells then enter the M phase in which the identical pairs of chromosomes are separated and transported to opposite ends of the cell. The cell then undergoes division into two identical daughter cells.

In a process known as spindle formation, the cell utilizes the mitotic spindle apparatus to separate and pull apart the chromosomes. This apparatus, in part, consists of a network of microtubules that form during the first stage of mitosis. Microtubules are hollow tubes that are formed by the assembly of tubulin heterodimers from alpha- and beta-tubulin. The assembly of tubulin into microtubules is a dynamic process with tubulin molecules being constantly added and subtracted from each end.

### Vinca Compounds as Inhibitors of Mitosis and Cellular Proliferation

In general, vinca compounds are known to be inhibitors of mitosis and cellular proliferation. In particular, the antiproliferative activity of the vinca alkaloid class of drugs has been shown to be due to their ability to bind tubulin. Assembly of tubulin into microtubules is essential for mitosis and the binding of the vincas to tubulin leads to cell cycle arrest in M phase and subsequently to apoptosis. For example, at low concentrations, these compounds interfere with the dynamics of microtubule formation. At higher concentrations, they cause microtubule disassembly, and at still higher concentrations, the formation of tubulin paracrystals.

Moreover, the anti-cancer activity of vinca alkaloids is generally believed to result from a disruption of microtubules resulting in mitotic arrest. However, cytotoxicity of vinca alkaloids also has been demonstrated in non-mitotic cells. Considering the role of microtubules in many cellular processes, the cytotoxic action of vinca alkaloids may involve contributions from inhibition of non-mitotic microtubule-dependent processes.

Cytotoxicity may also be a consequence of changes in membrane structure resulting from the partitioning of vinca alkaloids into the lipid bilayer. Studies with another tubulin binding compound, taxol, have shown that cell cycle arrest was not a precondition for apoptosis by agents of this type. Therefore, the anti-cancer activity of vinca alkaloids may be the result from disruption of a number of distinct microtubule-dependent and possibly microtubule-independent processes.

The assembly of tubulin into microtubules is a complex process involving dynamic instability (i.e. the switching between periods of slow growth and rapid shortening at both ends of the microtubule), and treadmilling (i.e. the addition of tubulin to one end of the microtubule occurring at the same rate as loss of tubulin from the other). Low concentrations of vinca alkaloids have been shown to bind to the ends of the microtubules and suppress both microtubule instability and treadmilling during the metaphase stage of mitosis. For example, vinca alkaloids have been shown to stabilize microtubule plus ends and destabilize microtubule minus ends. Although the spindle is retained under these conditions, there is frequently abnormal alignment of condensed chromosomes. At higher concentrations of vinca alkaloids, the spindle is not present and the chromosome distribution resembles that of prometaphase cells. At both low and high concentrations of vincas, mitotic arrest results from activation of metaphase-anaphase checkpoint. The molecular basis of this checkpoint is a negative signal sent from the kinetochore of chromosomes that are not attached to microtubules. This signal prevents the activation of pathways that result in the initiation of anaphase events.

Although there is a common binding site for the vinca alkaloids on tubulin, the members of this class do behave differently. The relative overall affinities for β-tubulin binding are vincristine>vinblastine>vinorelbine>vinflunine, but there is no significant difference in the affinity of all four drugs for tubulin heterodimers. The discrepancy has primarily been explained by differences in the affinities of vinca-bound heterodimers for spiral polymers and the binding of drug to unliganded polymers. For example, tubulin spirals induced by vinflunine are significantly smaller than those induced by vinorelbine.

In addition, vinca alkaloids also differ in their effects on microtubule dynamics. Vinflunine and vinorelbine suppress dynamic instability through: slowing the microtubule growth rate, increasing the mean duration of a growth event and reducing the duration of shortening. In contrast, vinblastine reduces the rate of shortening and increases the percentage of time the microtubules spend in the attenuated state. Vinblastine, vinorelbine, and vinflunine all suppress treadmilling, with vinblastine displaying the greatest potency.

### In Vivo Properties

The vinca derivatives fall into the general class of cytotoxic anti-cancer agents and, as such, suffer from the same problem as all cytotoxics—i.e., toxicity. Vincristine and vinblastine are neurotoxic. Vinorelbine, which is structurally very similar to vinblastine and vincristine and is only slightly less potent, is less neurotoxic. This change in toxicity cannot be explained by examination of the binding affinity of these compounds for tubulin alone. It has been postulated to arise from an increase in sensitivity to changes in microtubule dynamics in tumor cells and, as described above, these compounds have been shown to have subtly different effects. It could also arise from changes in cellular uptake of the drug. Vinflunine is not very potent in vitro yet is active in vivo, and this has been attributed to its superior cellular uptake. There are also quite significant differences in the profile of efficacy of vinca alkaloids. Vincristine has found wide use in the treatment of hematologic malignancies including leukemias and lymphomas. It is also widely used in pediatric solid tumors and, in the past, in small cell lung cancer. Vinblastine is an important component of the combination regimen that is curative for testicular cancer. Vinorelbine is quite different and has found use mainly in breast cancer and non-small cell lung cancer.

There remains a need for novel vinca derivatives with improved pharmacological and therapeutic properties, improved processes for the preparations of such vinca derivative compounds, corresponding pharmaceutical compositions, and methods of use.

The present invention is directed to achieving these objectives.

### SUMMARY OF THE INVENTION

The present invention relates to a compound of Formula (I) as follows:

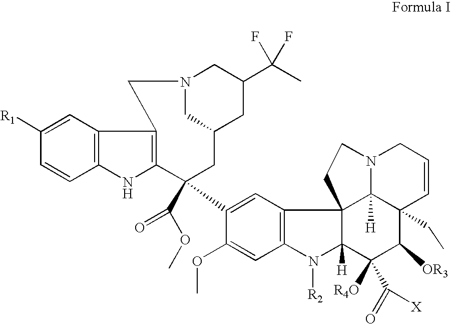

where:
R₁=alkyl;

alkenyl;

alkynyl;

aryl;

heterocyclyl;

halogen;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring

OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted.

Another compound in accordance with the present invention is the compound of Formula (II) as follows:

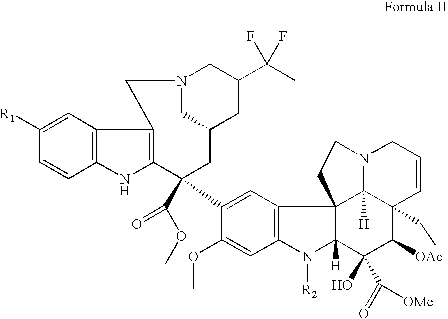

where:

### R₁ is

alkyl;

alkenyl;

alkynyl;

CN;

SR₅;

CF₃;

OR₇;

### R₂=alkyl or CH(O);

R₅ and R₇ are each independently hydrogen, alkyl, alkenyl, alkynyl, aryl, or heterocyclyl; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted.

A further compound pursuant to the present invention is the compound of Formula (III) as follows:

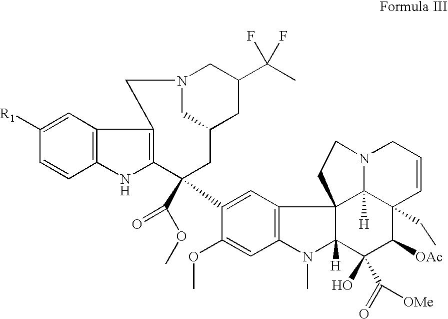

where:

### R₁ is:

alkyl;

SR₅;

OR₇;

R₅ and R₇ are each independently hydrogen, alkyl, alkenyl, alkynyl, aryl, or heterocyclyl; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted.

Another compound of the present invention is the compound of Formula (IV) as follows:

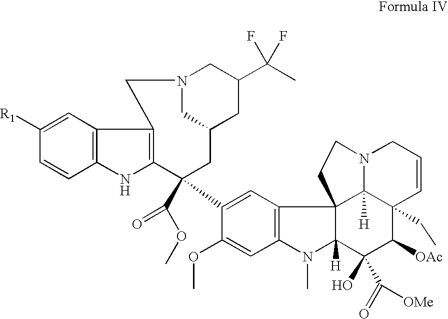

where:

### R₁ is alkyl which is substituted, unsubstituted, branched, or straight.

A further compound in accordance with the present invention is the compound of Formula (V) as follows:

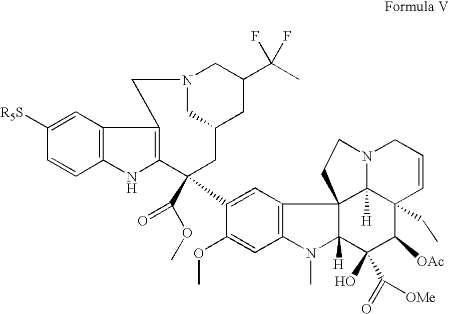

where:

### R₅=alkyl which is substituted, unsubstituted, branched, or straight.

Another aspect of the present invention relates to a process for preparation of a derivative product compound of Formula (I) as follows:

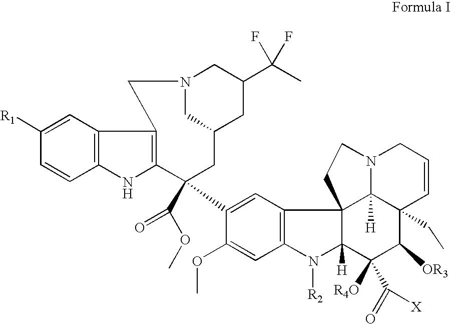

where:
R₁ is:

alkyl;

alkenyl;

alkynyl;

aryl;

heterocyclyl;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring;

OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process involves converting an intermediate compound of formula:

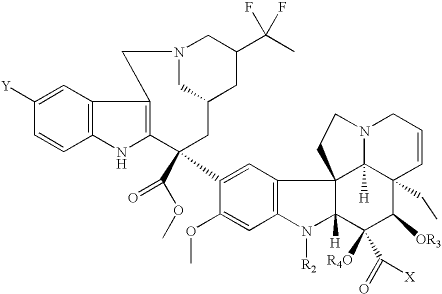

where Y is a halogen,
under conditions effective to produce the product compound of Formula (I).

Another aspect of the present invention relates to a process for preparation of a derivative product compound of Formula (I) as follows:

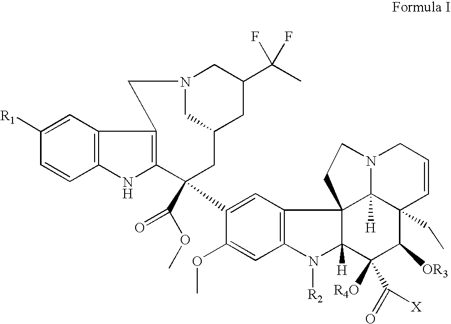

where:
R₁ is:

halogen;
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process involves halogenating a starting material of the formula:

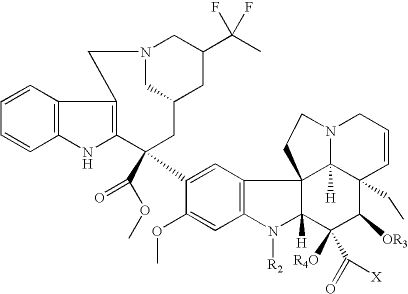

under conditions effective to form the derivative product compound.

A further aspect of the present invention relates to a process for preparation of a derivative product compound of Formula (I) as follows:

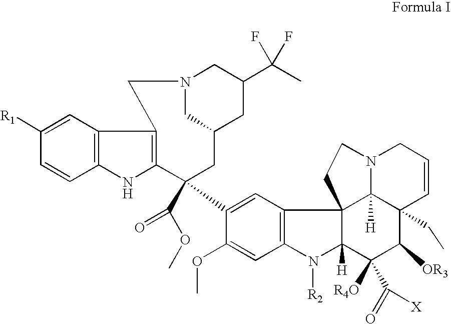

where:

### R₁ is:

alkyl;

alkenyl;

alkynyl;

aryl;

heterocyclyl;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring;

### R₂=alkyl or CH(O);

### R₃=hydrogen, alkyl, or C(O)R₅;

### R₄=hydrogen or C(O)R₅;

### R₅, R₆, and R₇ each are independently alkyl, alkenyl, alkynyl, aryl, or heterocyclyl;

### X=OR₅, NR₅R₆, NHNH₂, NHNHC(O)R₅, OH, NHR₅, NH₂, or NHNHC(O)H;

R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process includes converting a first intermediate compound of formula:

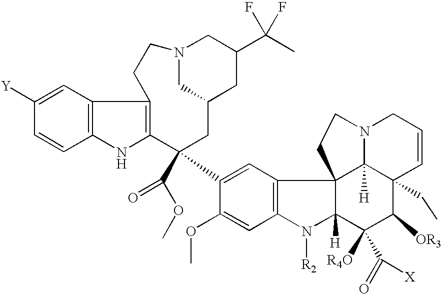

where Y is a halogen,
   under conditions effective to produce the product compound of Formula (I).

The present invention also relates to a method for inhibiting cell proliferation in mammals, which comprises administering a therapeutically effective amount of the compound of Formula (I) to the mammal.

The present invention also relates to a method for treating a condition in mammals, which comprises administering a therapeutically effective amount of the compound of Formula (I) to the mammal. The condition can be bacterial infection, allergy, heart disease, AIDS, Human T-lymphotropic virus 1 infection, Human herpesvirus 3, Human herpesvirus 4, Human papillomavirus, diabetes mellitus, rheumatoid arthritis, Alzheimer's Disease, inflammation, arthritis, asthma, malaria, autoimmune disease, eczema, Lupus erythematosus, psoriasis, rheumatic diseases, Sjogren's syndrome, and viral infection.

The present invention also relates to a pharmaceutical composition of matter, which comprises the compound of Formula (I) and one or more pharmaceutical excipients.

### DETAILED DESCRIPTION OF THE INVENTION

The present invention relates to novel vinorelbine derivatives, corresponding pharmaceutical compositions, preparation processes, and methods of use for treatment of various conditions.

In general, the novel compounds of the vinca family of compounds of the present invention, include derivatives of vinorelbine. In accordance with the present invention, such derivative compounds are represented by the chemical structures of Formula (I) as shown herein.

In particular, the present invention relates to a compound of Formula (I) as follows:

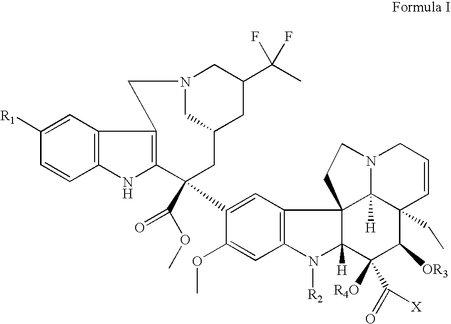

where:
R₁ is:

alkyl;

alkenyl;

alkynyl;

aryl;

heterocyclyl;

halogen;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring;

OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted.

As used above, and throughout the description of the invention, the following terms, unless otherwise indicated, shall be understood to have the following meanings:

The term “heterocyclyl” means the prefix aza, oxa, or thio before heterocycle means that at least a nitrogen, oxygen, or sulfur atom, respectively, is present as a ring atom. A nitrogen atom of a heteroaryl is optionally oxidized to the corresponding N-oxide. Representative monocyclic aromatic heterocycles include pyrrole, pyridine, oxazole, thiazole and the like. Representative monocyclic non-aromatic heterocycles include pyrrolidine, piperidine, piperazine and the like.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having 2 to about 6 carbon atoms in the chain. Preferred alkenyl groups have 2 to about 4 carbon atoms in the chain. Branched means that one or more lower alkyl groups such as methyl, ethyl or propyl are attached to a linear alkenyl chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having 2 to about 6 carbon atoms in the chain. Preferred alkynyl groups have 2 to about 4 carbon atoms in the chain. Branched means that one or more lower alkyl groups such as methyl, ethyl or propyl are attached to a linear alkynyl chain. Exemplary alkynyl groups include ethynyl, propynyl, n-butynyl, 2-butynyl, 3-methylbutynyl, and n-pentynyl.

The term “aryl” means an aromatic monocyclic or multicyclic ring system of 6 to about 14 carbon atoms, preferably of 6 to about 10 carbon atoms. Representative aryl groups include phenyl and naphthyl.

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded. “Unsubstituted” atoms bear all of the hydrogen atoms dictated by their valency. When a substituent is keto (i.e., ═O), then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

In one embodiment, the present invention relates to a compound where R₃=acetyl.

In another embodiment, the present invention relates to a compound where R₄=hydrogen.

In another embodiment, the present invention relates to a compound where X=OMe.

In another embodiment, the present invention relates to a compound where R₃=acetyl, R₄=hydrogen, and X=OMe.

In another embodiment, the present invention relates to a compound where R₂=CH(O).

In another embodiment, the present invention relates to a compound where R₂=alkyl.

### Representative examples of the compounds of Formula (I) are set forth in Table 1 below:

TABLE 1
Compounds of Formula (I)

| Example | | |
|---|---|---|
| Number | COMPOUND OF FORMULA (I) | NAME OF VINCA COMPOUND |
| 1 | 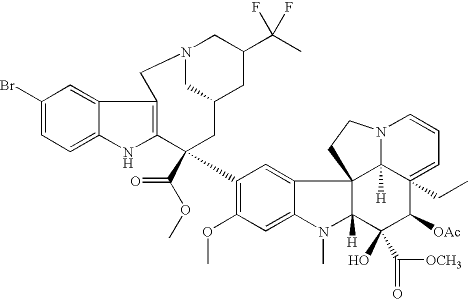 | 11′-Bromovinflunine |
| 2 | 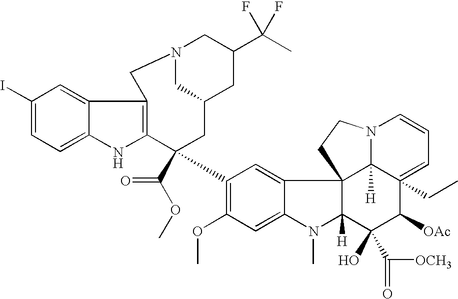 | 11′-Iodovinflunine |
| 3 | 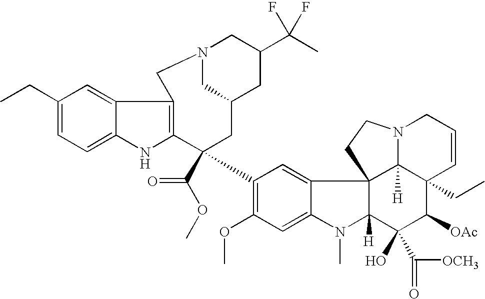 | 11′-Ethylvinflunine |
| 4 | 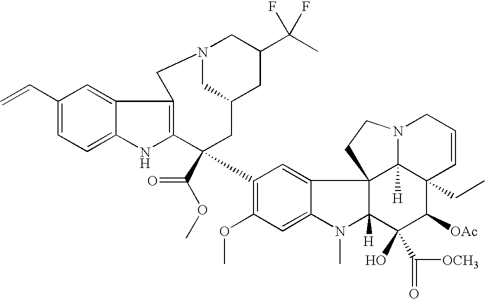 | 11′-Vinylvinflunine |
| 5 | 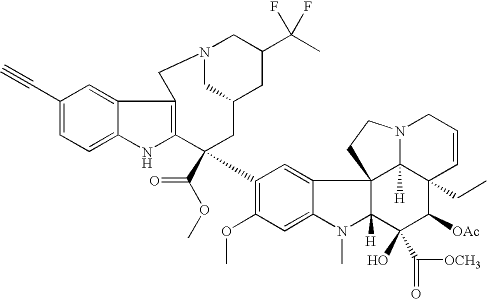 | 11′-Ethynylvinflunine |
| 6 | 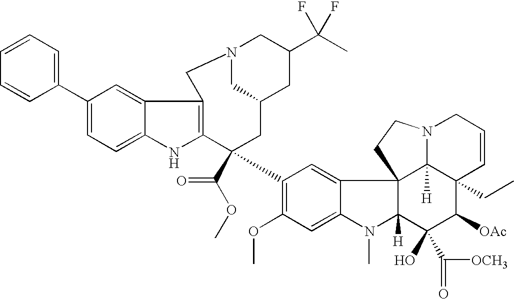 | 11′-Phenylvinflunine |
| 7 | 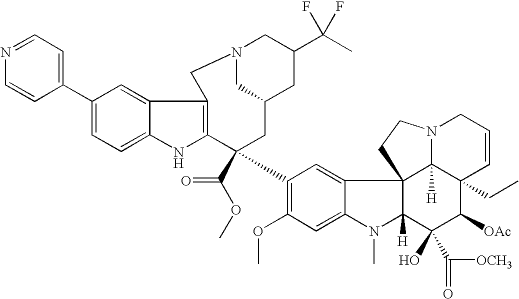 | 11′-(4-pyridyl)vinflunine |
| 8 | 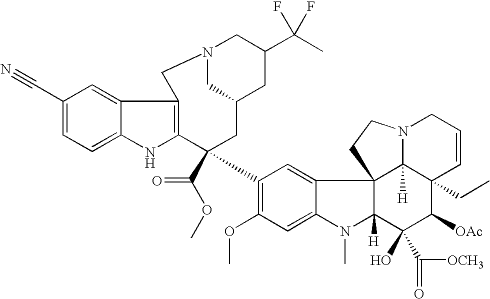 | 11′-Cyanovinflunine |
| 9 | 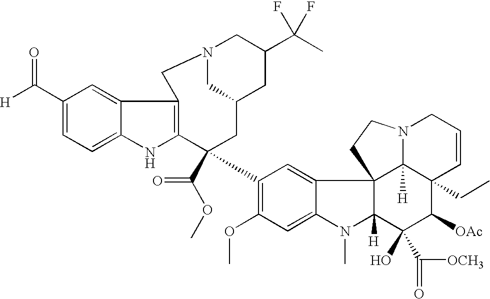 | 11′-Formylvinflunine |
| 10 | 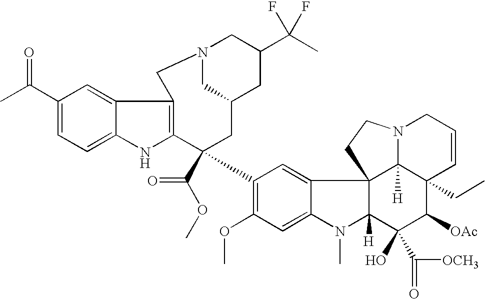 | 11′-Acetylvinflunine |
| 11 | 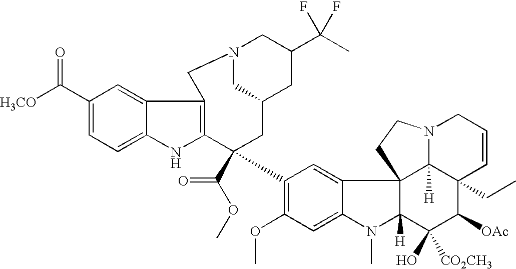 | 11′-(Methoxycarbonyl)vinflunine |
| 12 | 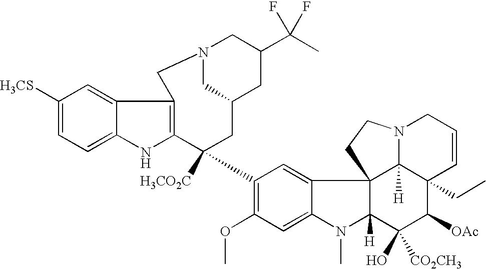 | 11′-(Methylsulfanyl)vinflunine |
| 13 | 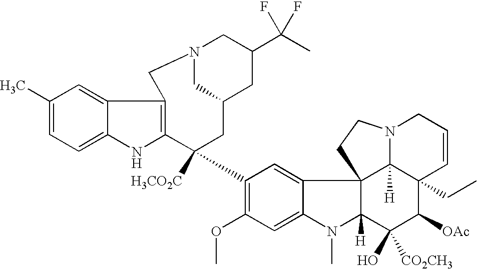 | 11′-Methylvinflunine |
| 14 | 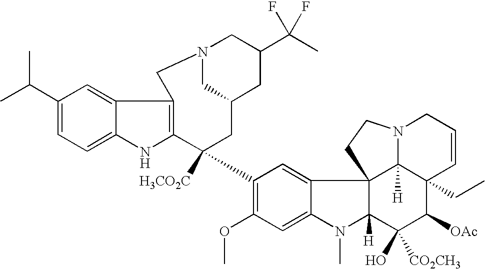 | 11′-Isopropylvinflunine |
| 15 | 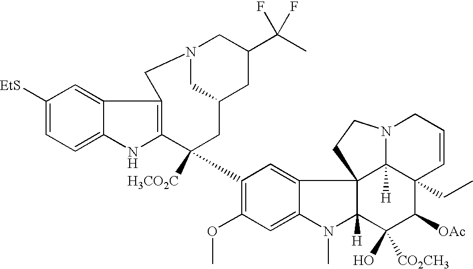 | 11′(Ethylsulfanyl)vinflunine |
| 16 | 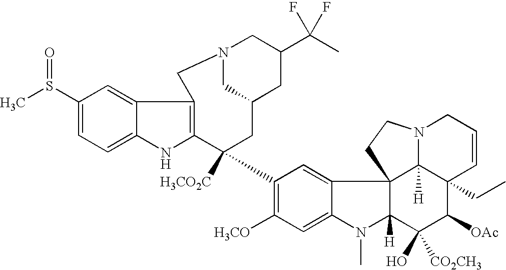 | 11′-(Methylsulfinyl)vinflunine |
| 17 | 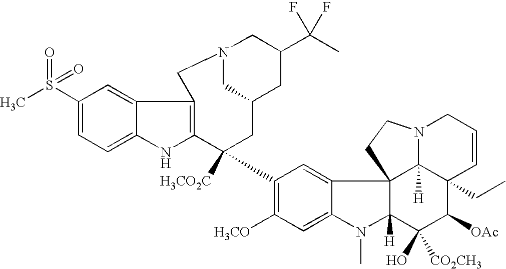 | 11′-(Methylsulfonyl)vinflunine |
| 18 | 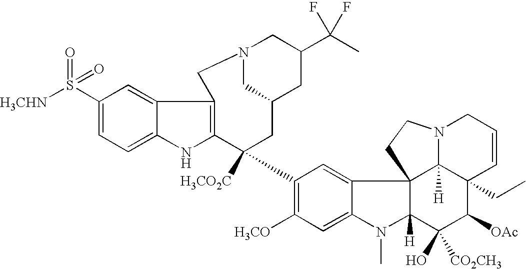 | 11′-(N-Methylsulfonamido)vinflunine |
| 19 | 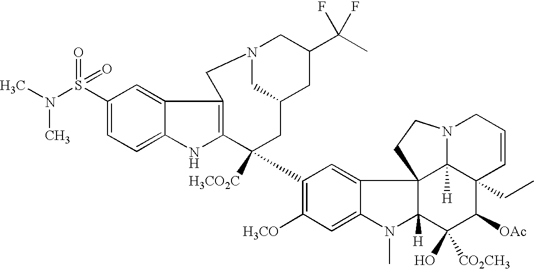 | 11′(N,N-Dimethylsulfonamido)vinflunine |
| 20 | 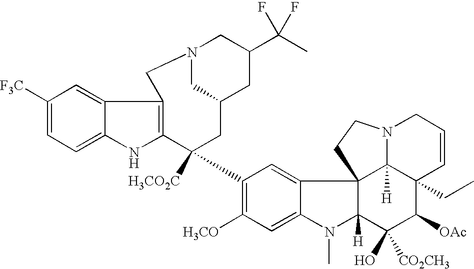 | 11′-Trifluoromethylvinflunine |
| 21 | 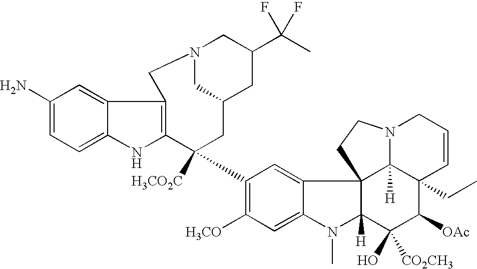 | 11′Aminovinflunine |
| 22 | 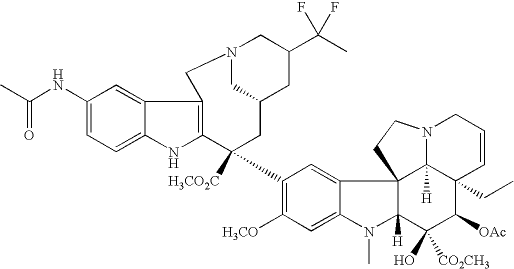 | 11′-Acetamidovinflunine |
| 23 | 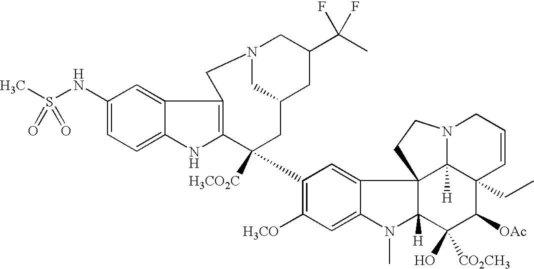 | 11′(Methanesulfonamido)vinflunine |
| 24 | 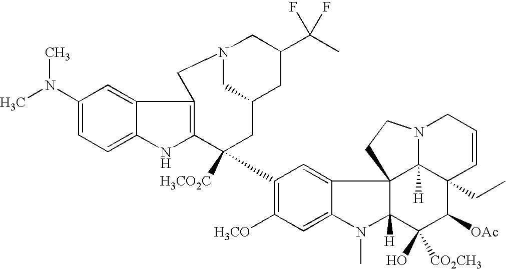 | 11′-(N,N-Dimethylamino)vinflunine |
| 25 | 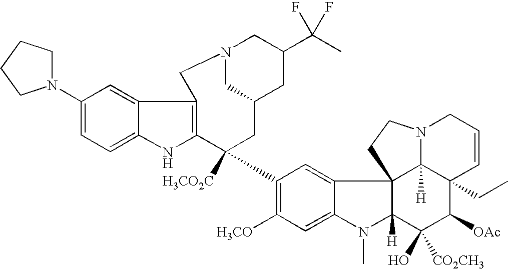 | 11′-(Pyrrolin-1-yl)vinflunine |
| 26 | 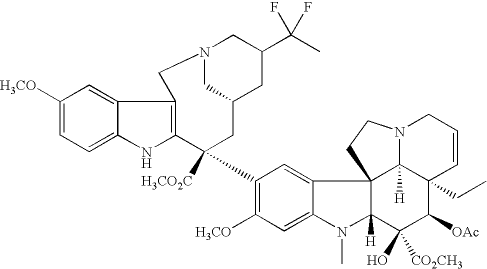 | 11′-Methoxyvinflunine |

In yet another embodiment of the present invention, a complex can be formed which includes 2 structures of Formula (I) joined together at their R₁ groups, wherein each R₁ is —S—.

Another compound in accordance with the present invention is the compound of Formula (II) as follows:

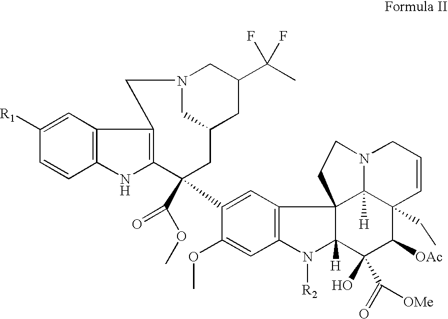

where:

### R₁ is

alkyl;

alkenyl;

alkynyl;

CN;

SR₅;

CF₃;

OR₇;

### R₂=alkyl or CH(O);

R₅ and R₇ are each independently hydrogen, alkyl, alkenyl, alkynyl, aryl, or heterocyclyl; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted.

A further compound pursuant to the present invention is the compound of Formula (III) as follows:

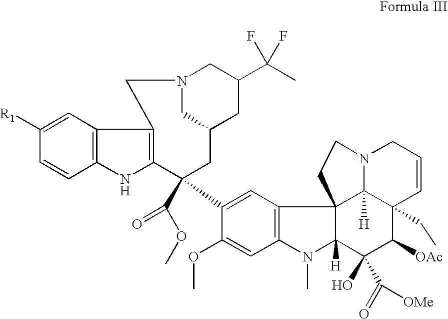

where:

### R₁ is:

alkyl;

SR₅;

OR₇;

R₅ and R₇ are each independently hydrogen, alkyl, alkenyl, alkynyl, aryl, or heterocyclyl; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted.

Another compound of the present invention is the compound of Formula (IV) as follows:

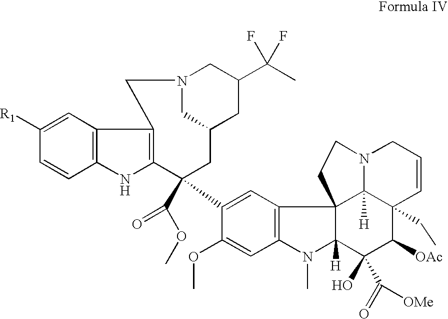

where:

### R₁ is alkyl which is substituted, unsubstituted, branched, or straight.

Example of compounds of Formula IV are:

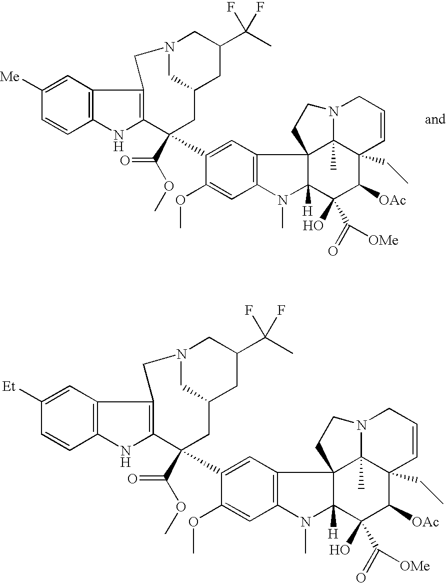

A further compound in accordance with the present invention is the compound of Formula (V) as follows:

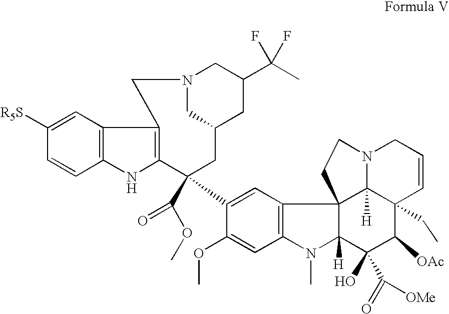

where:

### R₅=alkyl which is substituted, unsubstituted, branched, or straight.

Examples of compounds of Formula V are:

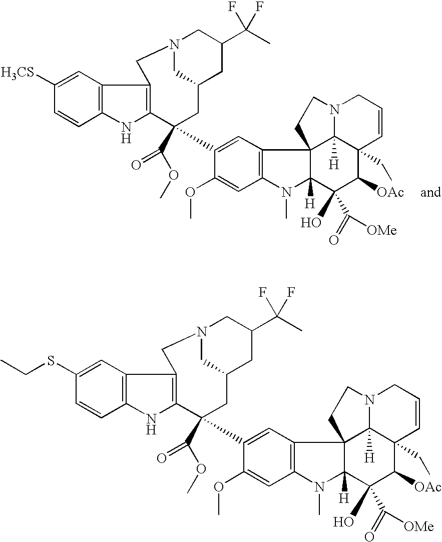

Another aspect of the present invention relates to a process for preparation of a derivative product compound of Formula (I) as follows:

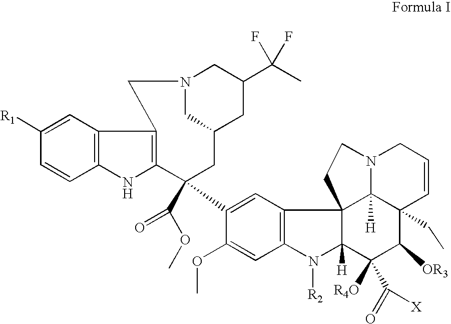

where:
R₁ is:

alkyl;

alkenyl;

alkynyl;

aryl;

heterocyclyl;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring;

OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process involves converting an intermediate compound of formula:

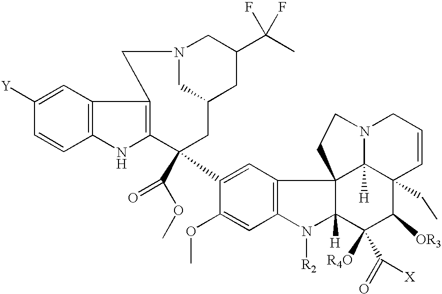

where Y is a halogen,
under conditions effective to produce the product compound of Formula (I).

The intermediate compound is formed by halogenating a starting material compound of formula:

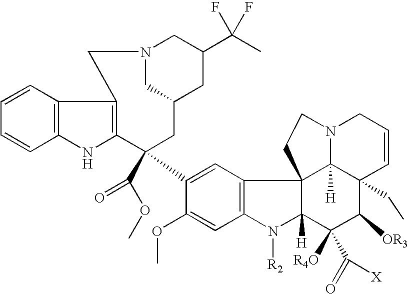

under conditions effective to form the intermediate compound.

Another aspect of the present invention relates to a process for preparation of a derivative product compound of Formula (I) as follows:

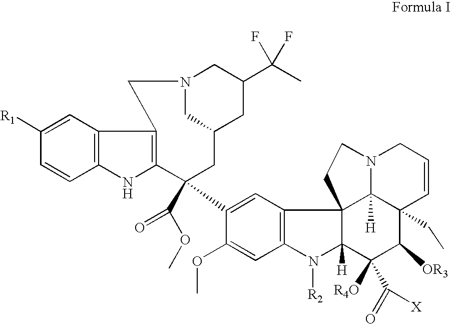

where:

### R₁ is:

halogen;

### R₂=alkyl or CH(O);

### R₃=hydrogen, alkyl, or C(O)R₅;

### R₄=hydrogen or C(O)R₅;

### R₅, R₆, and R₇ each are independently alkyl, alkenyl, alkynyl, aryl, or heterocyclyl;

### X=OR₅, NR₅R₆, NHNH₂, NHNHC(O)R₅, OH, NHR₅, NH₂, or NHNHC(O)H;

R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process involves halogenating a starting material of the formula:

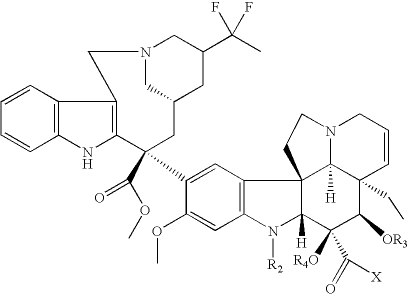

under conditions effective to form the derivative product compound.

A further aspect of the present invention relates to a process for preparation of a derivative product compound of Formula (I) as follows:

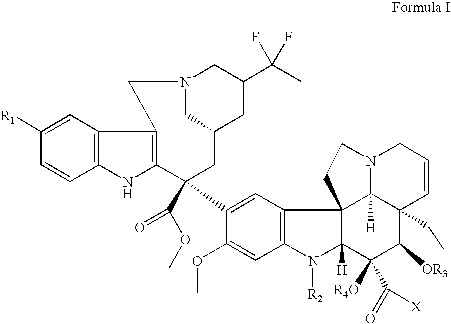

where:
R₁ is:

alkyl;

alkenyl;

alkynyl;

aryl;

heterocyclyl;

CN;

CH(O);

COR₅;

SO₂NHNH₂;

SO₂NR₅NH₂;

SO₂NR₅NHR₆;

SO₂NR₅NR₆R₇;

SO₂NHNHR₅;

SO₂NHNR₅R₆;

CO₂R₅;

SR₅;

SSR₅;

SOR₅;

SO₂R₅;

SO₂NHR₅;

SO₂NR₅R₆;

B(OR₅)₂;

CF₃;

SH;

SO₂NH₂;

NH₂;

NHR₅;

NHCOR₅;

NHSO₂R₅;

NR₅R₆;

NR₅COR₆; or

NR₅SO₂R₆;

R₅ and R₆ can form a ring;
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
   R₄ and X may be linked together with intervening atoms to form a ring; or a pharmaceutically acceptable salt thereof, where the alkyl and alkenyl groups may be branched, straight, unsubstituted, and/or substituted and where the aryl, alkynyl, and heterocyclyl groups are substituted or unsubstituted. The process includes converting a first intermediate compound of formula:

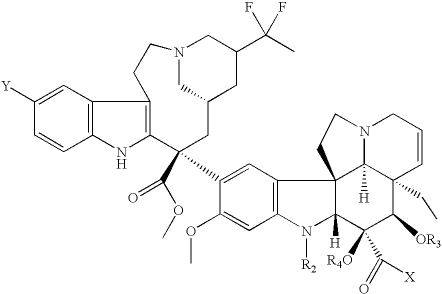

where Y is a halogen,
   under conditions effective to produce the product compound of Formula (I).

The first intermediate compound is produced by fluorinating a second intermediate compound of the formula:

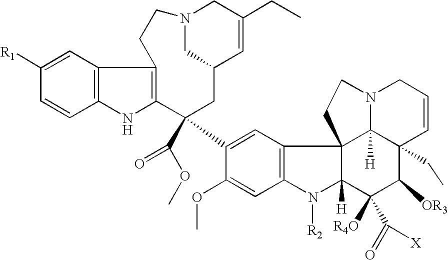

under conditions effective to procude the first intermediate compound.

The second intermediate compound is formed by converting a third intermediate compound of the formula:

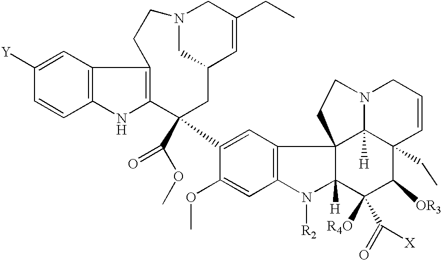

where Y is a halogen,
   under conditions effective to form the second intermediate compound.

The third intermediate compound is formed by halogenating a starting material of the formula:

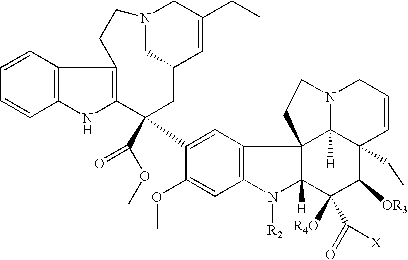

under conditions effective to produce the third intermediate compound.

The synthetic reaction schemes for the preparation of compounds of Formula (I) are depicted below.

A synthetic scheme for preparing compounds of Formula (I) is shown in Scheme 1 below. A vinca alkaloid is treated with either N-iodosuccinimide to introduce an iodine in the 11′-position or subjected to enzymatic bromination to introduce a bromine in the 11′-position. Pd-mediated coupling is then used to introduce other functionality at this position. This methodology can be used to introduce alkyl, alkenyl, alkynyl, aryl, heterocyclyl, acyl, and formyl groups and to form sulphides. Each of these groups can then be subjected to further derivitization following stand methods of organic synthesis.

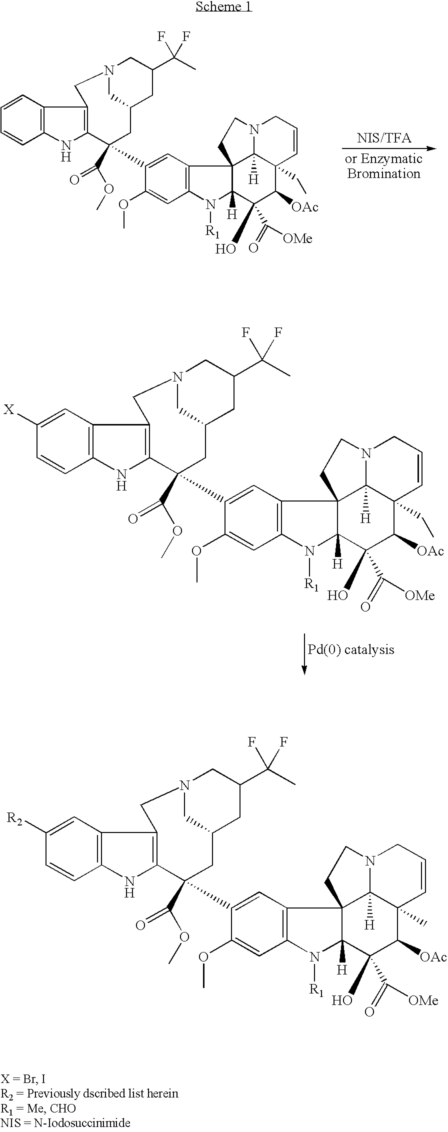

Scheme II shows an alternative synthesis starting from anhydrovinblastine.

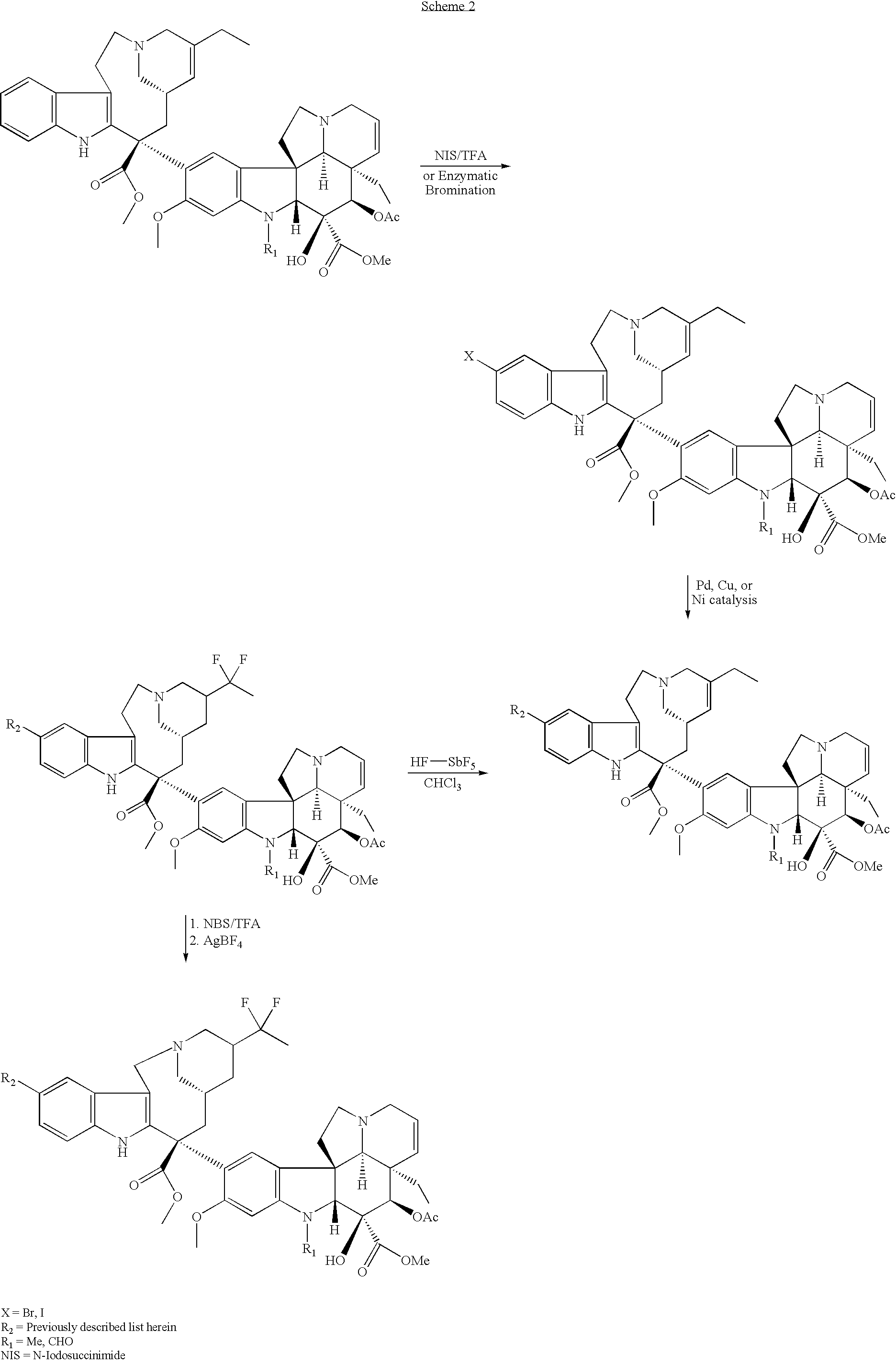

In practicing either of the above processes, a variety of catalysts may be utilized, such as palladium chloride, palladium acetate, tetrakis(triphenylphosphine)palladium(0), tris(dibenzylideneacetone)dipalladium(0), dichlorobis(triphenylphosphine)palladium(II), benzylchlorobis(triphenylphosphine)palladium(II), [1,1′-bis(diphenylphosphino)ferrocene]dichloropalladium(II), tetrakis(triphenylphosphine)palladium, or bis(triphenylphosphine) palladium(II)dichloride. Additionally, the catalyst reactivity can be modified by addition of appropriate ligands or additives. Representative ligands or additives include: 2-(dicyclohexylphosphino)-2′,4′,6′-tri-1-propyl-1,1′-biphenyl, 2-(dicyclohexylphosphino)-2′,6′-dimethoxy-1,1′-biphenyl, PPh₃, t-Bu₃P, CuI, or CuBr.

The compounds of the present invention are useful in inhibiting cellular proliferation in a mammal by administering to such mammal an effective amount of compound(s) of the present invention.

In particular, such vinca derivatives are useful as antineoplastic agents. More particularly, the compounds of the present invention are useful for inhibiting the growth of neoplastic cells, causing cell death of neoplastic cells, and eradicating neoplastic cells. The compounds of the present invention are, therefore, useful for treating solid tumors, (e.g., sarcomas), carcinomas, (e.g., astrocytomas), lymphomas, (e.g., adult T-cell lymphoma), different cancer disease types, (e.g., prostate cancer, breast cancer, small cell lung cancer, ovarian cancer, (Hodgkin's Disease), and other neoplastic disease states (e.g., leukemias, particularly adult T-cell leukemias).

Since vinca compounds are known to be tubulin inhibitors, the compounds of the present invention would also be expected to be useful in treating the following conditions: bacterial infection; allergy; heart disease; AIDS; Human T-lymphotropic virus 1 infection; Human herpesvirus 3; Human herpesvirus 4; Human papillomavirus; diabetes mellitus; rheumatoid arthritis; Alzheimer's Disease; inflammation; arthritis; asthma; malaria; autoimmune disease; eczema; Lupus erythematosus; psoriasis; rheumatic diseases; Sjogren's syndrome; and viral infection.

The vinca derivatives of the present invention can be administered alone as indicated above, or utilized as biologically active components in pharmaceutical compositions with suitable pharmaceutically acceptable carriers, adjuvants and/or excipients.

In accordance with the present invention, the compounds and/or corresponding compositions can be introduced via different administration routes, which include orally, parenterally, intravenously, intraperitoneally, by intranasal instillation, or by application to mucous membranes, such as, that of the nose, throat, and bronchial tubes.

The active compounds of the present invention may be orally administered, for example, with an inert diluent, or with an assimilable edible carrier, or they may be enclosed in hard or soft shell capsules, or they may be compressed into tablets.

The quantity of the compound administered will vary depending on the patient and the mode of administration and can be any effective amount. The quantity of the compound administered may vary over a wide range to provide in a unit dosage an effective amount of from about 0.01 to 20 mg/kg of body weight of the patient per day to achieve the desired effect. The amount of active compound in such therapeutically useful compositions is such that a suitable dosage will be obtained. Preferred compositions according to the present invention are prepared so that an oral dosage unit contains between about 1 and 250 mg of active compound.

For example, with oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both.

These active compounds and/or pharmaceutical compositions may also be administered parenterally. Solutions of these active compounds and/or compositions can be prepared in water. Dispersions can also be prepared in glycerol, liquid polyethylene glycols, and mixtures thereof in oils.

Illustrative oils are those of animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the pharmaceutical form of the present invention must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds and/or pharmaceutical compositions of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Some of the compounds of the present invention can be in the form of pharmaceutically acceptable acid-addition and/or base salts. All of these forms of salts are within the scope of the present invention.

Pharmaceutically acceptable acid addition salts of the compounds of the present invention include salts derived from nontoxic inorganic acids, such as hydrochloric acid, nitric acid, phosphoric acid, sulfuric acid, hydrobromic acid, hydroiodic acid, hydrofluoric acid, phosphorous acid, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety).

The acid addition salts of said basic compounds are prepared by contacting the free base forms with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N-dibenzylethylenediamine, chloroprocaine, choline, diethanolamine, dicyclohexylamine, ethylenedianline, N-methylglucamine, and procaine (see, for example, Berge S. M. et al., “Pharmaceutical Salts,” Journal of Pharmaceutical Science, 66:1-19 (1997), which is hereby incorporated by reference in its entirety).

The base addition salts of the acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

The present invention can be used in conjunction with other known cancer treatments, including other chemotherapeutic agents and radiation.

### EXAMPLES

Compounds in accordance with the present invention are made pursuant to Scheme 3 as follows:

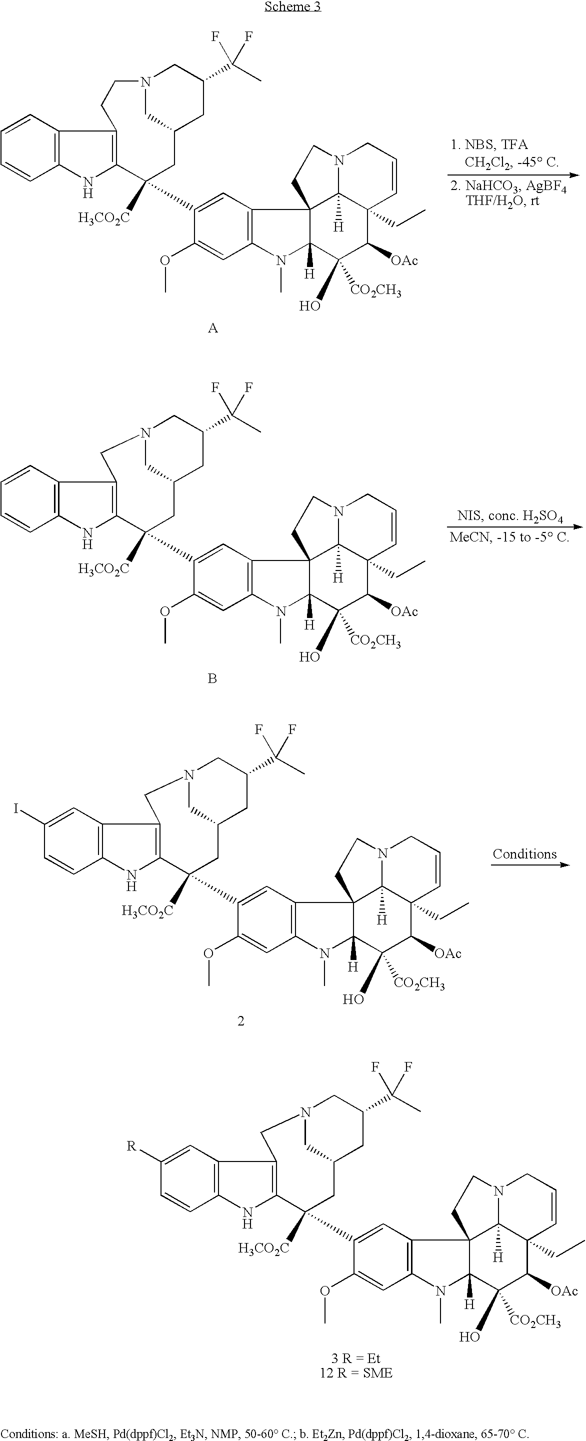

### Example 1

### Preparation of 20′, 20′-Difluoro-3′,4′-dihydrovinorelbine (B)

This compound was prepared from 4′-deoxy-20′, 20′-difluorovinblastine (A) according to a reported procedure (U.S. Pat. No. 6,127,377, which is hereby incorporated by reference in its entirety), the spectral data of which were consistent with those reported therein.

### Example 2

### Preparation of 20′,20′-Difluoro-3′,4′-dihydro-12′-iodovinorelbine (2)

20′,20′-difluoro-3′,4′-dihydrovinorelbine (B; bis-TFA salt; 250 mg, 0.24 mmol) was dissolved in MeCN (2 mL) and cooled to −15° C. Then conc. H₂SO₄ (0.30 mL) was added dropwise at −15° C. followed by dropwise addition of NIS (0.24 mmol) in MeCN (1 mL) at −10˜−15° C. The reaction mixture was stirred at −15˜−10° C. for 2 h, quenched with saturated NaHCO₃ (10 mL) and extracted with EtOAc (3×10 mL). The combined extracts were dried over Na₂SO₄, filtered, and concentrated to give the crude title compound as a brown-yellow solid. Purification by column chromatography gave a white solid (134 mg, 59%): ¹H NMR (500 MHz, CDCl₃) δ 9.72 (s, 1H), 8.43 (s, 1H), 7.97 (s, 1H), 7.40 (d, J=8.3 Hz, 1H), 6.91 (d, J=8.4 Hz, 1H), 6.28 (s, 1H), 6.07 (s, 1H), 5.86-5.84 (m, 1H), 5.39 (s, 1H), 5.30-5.26 (m, 2H), 4.47 (d, J=12.6 Hz, 1H), 4.21 (d, J=12.6 Hz, 1H), 3.80 (s, 3H), 3.78 (s, 3H), 3.71 (s, 4H), 3.36-3.25 (m, 4H), 3.06-2.91 (m, 2H), 2.84-2.72 (m, 2H), 2.70 (s, 3H), 2.56 (br d, J=9.6 Hz, 1H), 2.47 (s, 1H), 2.35-2.34 (m, 1H), 2.24 (br d, J=14.7 Hz, 1H), 2.13-2.04 (m, 5H), 1.81-1.75 (m, 2H), 1.73-1.68 (m, 1H), 1.13-1.22 (m, 2H), 1.11 (s, 1H), 0.65 (t, J=7.1 Hz, 3H); ES-MS: (M+H)=943 m/z.

### Example 3

### Preparation of 20′, 20′-Difluoro-3′,4′-dihydro-12′-methylthiovinorelbine (12)

This compound was prepared from 20′, 20′-difluoro-3′,4′-dihydro-12′-iodovinorelbine (2; 130 mg, 0.14 mmol)) and Pd(dppf)Cl₂ (0.028 mmol) in NMP (2 mL) the mixture was degassed and purged with methanethiol three times. The reaction mixture was heated at 65° C. for 46 h. LC-MS analysis showed completion of the reaction. The reaction mixture was cooled to room temperature, diluted with EtOAc (15 mL) and washed with water (10 mL). The aqueous layer was extracted with EtOAc (2×10 mL). The combined organic layers were extracted with 0.5 N HCl (4×10 mL). The combined aqueous extracts were neutralized with NaHCO₃ and extracted with EtOAc (4×15 mL). The combined organic layers were dried over Na₂SO₄ and concentrated. After purification by preparative HPLC and conversion to ditartrate salt, lyophilization gave a white solid (32 mg, 20%): ¹H NMR (500 MHz, D₂O) δ 7.66 (s, 1H), 7.40 (d, J=8.6 Hz, 1H), 7.27 (dd, J=8.6, 1.4 Hz, 1H), 6.42 (s, 1H), 6.41 (s, 1H), 5.90 (dd, J=10.4, 5.3 Hz, 1H), 5.62 (d, J=10.9 Hz, 1H), 5.22 (s, 1H), 5.00 (d, J=15.0 Hz, 1H), 4.86-4.82 (m, 2H), 4.43 (s, 4.0H), 3.88-3.85 (m, 1H), 3.82 (s, 3H), 3.81 (s, 3H), 3.71 (s, 3H), 3.78-3.66 (m, 3H), 3.56 (s, 1H), 3.49 (d, J=12.7 Hz, 1H), 3.43-3.33 (m, 2H), 3.16-2.98 (m, 3H), 2.86 (d, J=12.7 Hz, 1H), 2.70 (s, 3H), 2.65 (dd, J=15.9, 6.4 Hz, 1H), 2.50 (s, 3H), 2.41-2.35 (m, 1H), 2.07 (s, 3H), 1.95-1.86 (m, 2H), 1.70-1.57 (m, 5H), 1.52 (br s, 1H), 1.32-1.28 (m, 1H), 0.63 (t, J=7.2 Hz, 3H); ES-MS: (M+H)=863 m/z; HPLC t_{R}=12.05 min, 98.8%. Calcd for C₄₆H₅₆F₂N₄O₈S.2C₄H₆O₆.2H₂O: <<Calcd>> <<Found>>

### Example 4

### Preparation of 20′,20′-Difluoro-3′,4′-dihydro-12′-ethylvinorelbine (3)

A mixture of 20′, 20′-difluoro-3′,4′-dihydro-12′-iodovinorelbine (2; 345 mg, 0.36 mmol) and Et₂Zn (0.50 ml, 3.6 mmol) in 1,4-dioxane (5 mL) was degassed and purged with N₂ three times. Then, Pd(dppf)Cl₂ (60 mg, 0.072 mmol) was added and the reaction mixture was purged with N₂. The reaction mixture was heated at 80-90° C. for 6.5 h, cooled to room temperature, quenched with saturated aqueous NaHCO₃ (5 mL) and extracted with EtOAc (4×5 mL). The combined organic layers were dried over Na₂SO₄ and concentrated. After purification by column chromatography followed by preparative HPLC and conversion to ditartrate salt, lyophilization gave a white solid (41 mg, 10%): ¹H NMR (500 MHz, D₂O) δ 7.53 (s, 1H), 7.37 (d, J=8.4 Hz, 1H), 7.18 (d, J=8.4 Hz, 1H), 6.42 (s, 1H), 6.38 (s, 1H), 5.90 (dd, J=10.4, 4.4 Hz, 1H), 5.62 (d, J=10.0 Hz, 1H), 5.23 (s, 1H), 5.00 (d, J=15.0 Hz, 1H), 4.85 (d, J=15.0 Hz, 1H), 4.44 (s, 5.0H), 3.88-3.81 (m, 1H), 3.83 (s, 3H), 3.81 (s, 3H), 3.72 (s, 6H), 3.52 (s, 1H), 3.47 (d, J=13.0 Hz, 1H), 3.40 (t, J=13.0 Hz, 1H), 3.34 (d, J=15.6 Hz, 1H), 3.24-3.15 (m, 1H), 3.08-2.98 (m, 2H), 2.85 (d, J=13.8 Hz, 1H), 2.74-2.65 (m, 3H), 2.69 (s, 3H), 2.41-2.35 (m, 1H), 2.07 (s, 3H), 1.96-1.87 (m, 2H), 1.72-1.57 (m, 2H), 1.66 (t, J=19.6 Hz, 3H), 1.50 (br s, 1H), 1.32-1.25 (m, 1H), 1.20 (t, J=7.5 Hz, 4H), 0.65 (t, J=7.3 Hz, 3H); ES-MS: (M+H)=845 m/z; HPLC t_{R}=12.05 min, >99%. Calcd for C₄₇H₅₈F₂N₄O₈S.2.5C₄H₆O₆.3H₂O: <<Calcd>><<Found>>

### Example 5

### Description of Biological Assays

A. HeLa GI₅₀ Determinations

Growth inhibition (GI₅₀) values were measured on the human cervical carcinoma cell line, HeLa S-3, which were selected for growth on plastic. The HeLa cell assay was based on the description of Skehan et al., J. Natl. Cancer Inst., 82:1107-12 (1990), which is hereby incorporated by reference in its entirety. HeLa cells were plated at 2×10⁴ cells/well in 96 well plates. One day later, a control plate was fixed by the addition of TCA to 5%. After five rinses with tap water, the plate was air-dried and stored at 4° C. Test compounds were added to the remaining plates at 10-fold dilutions. Two days later, all plates were fixed as described above. Cells were then stained by the addition of 100 μL per well of 0.4% sulforhodamine B (SRB) in 1% acetic acid for 30 min at 4° C. Wells were then quickly rinsed 5× with 1% acetic acid and allowed to air dry. The SRB was then solubilized by the addition of 100 μL per well of unbuffered 10 mM Tris base. Dye was quantified by measuring absorbance at 490 nm on a Molecular Devices microplate reader. Growth inhibition was calculated according to the following equation: GI=100×(T−T₀)/(C−T₀), where the optical density (OD) of the test well after 2 days of treatment was T, the OD of the wells in the control plate on day 0 was T₀ and C was the OD of untreated wells. Plots of percent growth inhibition versus inhibitor concentration were used to determine the GI₅₀.

TABLE 2
Growth Inhibition (GI₅₀) of HeLa Cells
for Compounds of the Present Invention.

| | HeLa Cells |
|---|---|
| Example | GI₅₀ (nM) |
| 3 | 170 |
| 12 | 6.93 |

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A compound of Formula (I) as follows: 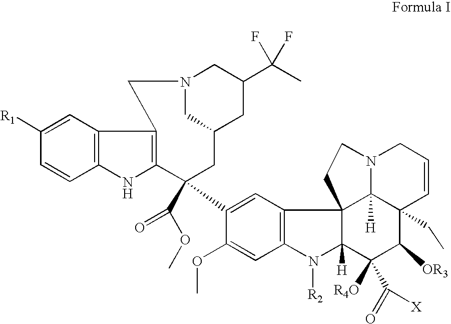
where:
R₁ is:
alkyl;
alkenyl;
alkynyl;
aryl;
heterocyclyl;
halogen;
CN;
CH(O);
COR₅;
SO₂NHNH₂;
SO₂NR₅NH₂;
SO₂NR₅NHR₆;
SO₂NR₅NR₆R₇;
SO₂NHNHR₅;
SO₂NHNR₅R₆;
CO₂R₅;
SR₅;
SSR₅;
SOR₅;
SO₂R₅;
SO₂NHR₅;
SO₂NR₅R₆;
B(OR₅)₂;
CF₃;
SH;
SO₂NH₂;
NH₂;
NHR₅;
NHCOR₅;
NHSO₂R₅;
NR₅R₆;
NR₅COR₆; or
NR₅SO₂R₆;
R₅ and R₆ can form a ring;
OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
R₄ and X may be linked together with intervening atoms to form a ring; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted.

2. The compound according to claim 1, wherein R₃=acetyl.

3. The compound according to claim 1, wherein R₄=hydrogen.

4. The compound according to claim 1, wherein X=OMe.

5. The compound according to claim 1, wherein R₃=acetyl, R₄=hydrogen, and X=OMe.

6. The compound according to claim 1, wherein R₂=CH(O).

7. The compound according to claim 1, wherein R₂=alkyl.

8. A compound of Formula II as follows: 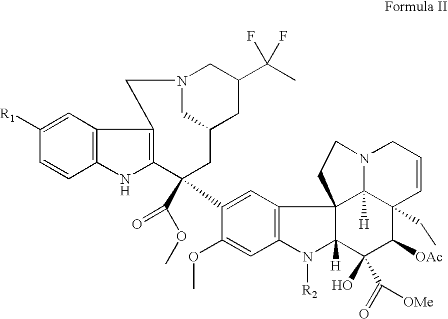
where:
R₁ is
alkyl;
alkenyl;
alkynyl;
CN;
SR₅;
CF₃;
OR₇;
R₂=alkyl or CH(O);
R₅ and R₇ are each independently hydrogen, alkyl, alkenyl, alkynyl, aryl, or heterocyclyl; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted

9. A compound of Formula III as follows: 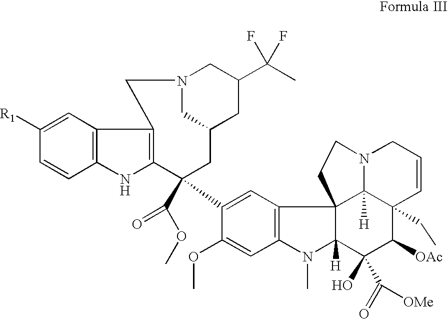
where:
R₁ is:
alkyl;
SR₅;
OR₇;
R₅ and R₇ are each independently hydrogen, alkyl, alkenyl, alkynyl, aryl, or heterocyclyl, or a pharmaceutically acceptable salt thereof, wherein the alkyl and alkenyl groups may be branched, straight, unsubstitutued, and/or substituted and wherein the aryl, alkynyl, and heterocyclyl groups are substituted or unsubstituted.

10. A compound of Formula IV as follows: 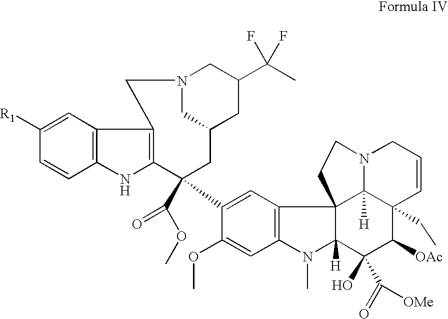
where:
R₁ is alkyl which is substituted, unsubstituted, branched, or straight.

11. The compound according to claim 10, wherein the compound has the following chemical formula:

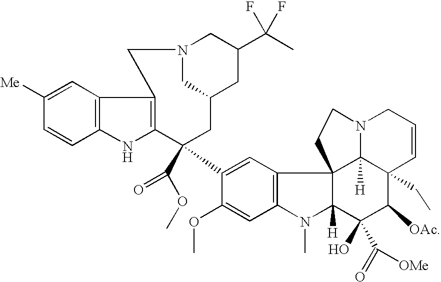

12. The compound according to claim 10, wherein the compound has the following chemical formula:

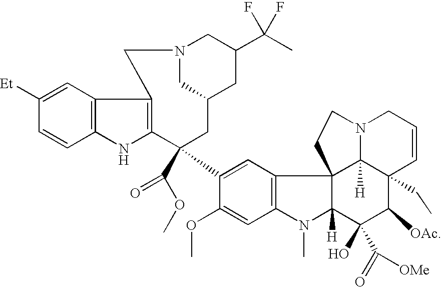

13. A compound of Formula V as follows: 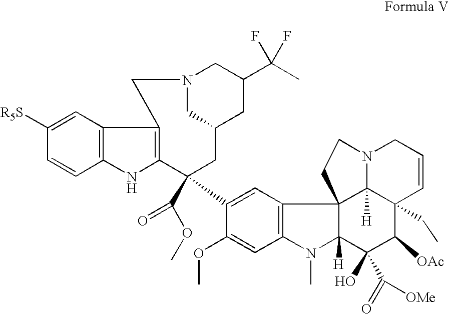
where:
R₅=alkyl which is substituted, unsubstituted, branched, or straight.

14. The compound according to claim 13, wherein the compound has the following chemical formula:

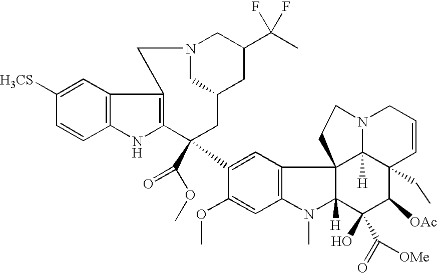

15. The compound according to claim 13, wherein the compound has the following chemical formula:

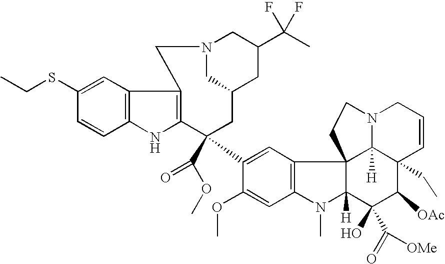

16. A process for preparation of a derivative product compound of Formula (I) as follows: 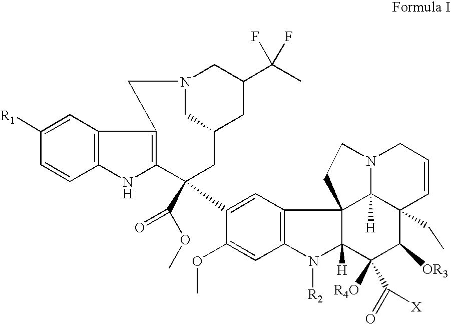
where:
R₁ is:
alkyl;
alkenyl;
alkynyl;
aryl;
heterocyclyl;
CN;
CH(O);
COR₅;
SO₂NHNH₂;
SO₂NR₅NH₂;
SO₂NR₅NHR₆;
SO₂NR₅NR₆R₇;
SO₂NHNHR₅;
SO₂NHNR₅R₆;
CO₂R₅;
SR₅;
SSR₅;
SOR₅;
SO₂R₅;
SO₂NHR₅;
SO₂NR₅R₆;
B(OR₅)₂;
CF₃;
SH;
SO₂NH₂;
NH₂;
NHR₅;
NHCOR₅;
NHSO₂R₅;
NR₅R₆;
NR₅COR₆; or
NR₅SO₂R₆;
R₅ and R₆ can form a ring;
OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
R₄ and X may be linked together with intervening atoms to form a ring; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted, said process comprising:
converting an intermediate compound of the formula: 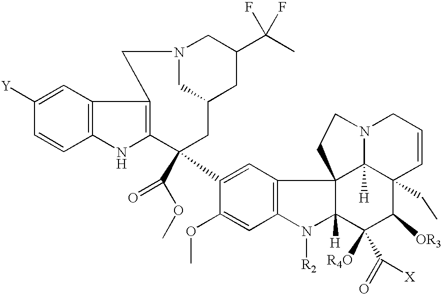
wherein Y is a halogen,
under conditions effective to produce the product compound of Formula (I).

17. The process of claim 16 further comprising:
halogenating a starting material compound of formula: 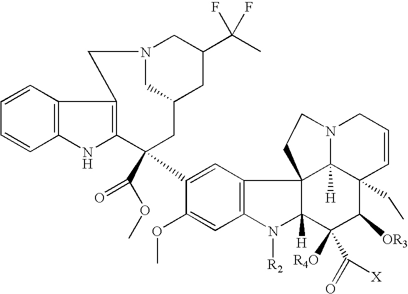
under conditions effective to form the intermediate compound.

18. The process of claim 17, wherein said halogenating is carried out with a halogenating agent selected from the group consisting of N-bromosuccinimide, N-iodosuccinimide, and iodine monochloride.

19. The process of claim 17, wherein the conditions effective to form the intermediate compound include enzymatic bromination.

20. The process of claim 16, wherein said converting comprises:
reacting the intermediate compound with a palladium catalyst reagent to produce the product of Formula (I).

21. The process of claim 20, wherein the palladium catalyst reagent is selected from the group consisting of palladium acetate, tris(dibenzylideneacetone)dipalladium(0), [1,1′-bis(diphenylphosphino)ferrocene]dichloropalladium(II), tetrakis(triphenylphosphine)palladium, and bis(triphenylphosphine) palladium(II)dichloride.

22. A process for preparation of a derivative product compound of Formula (I) as follows: 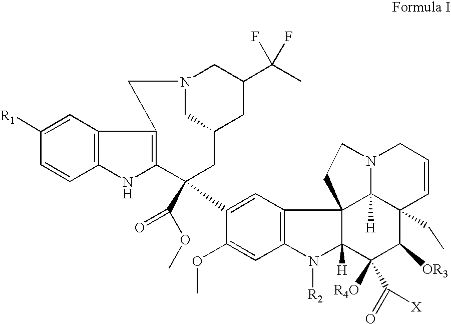
where:
R₁ is:
halogen;
R₂=alkyl or CH(O);
R₃=hydrogen, alkyl, or C(O)R₅;
R₄=hydrogen or C(O)R₅;
R₅ and R₆ each are independently alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
R₄ and X may be linked together with intervening atoms to form a ring; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted, said process comprising:
halogenating a starting material compound of formula: 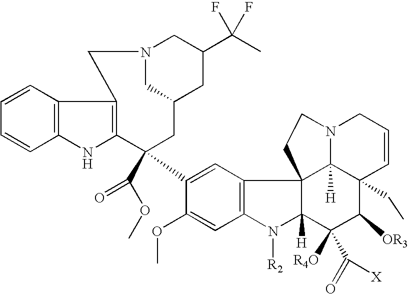
under conditions effective to form the derivative product compound.

23. The process of claim 22, wherein said halogenating is carried out with a halogenating agent selected from the group consisting of N-bromosuccinimide, N-iodosuccinimide, and iodine monochloride.

24. The process of claim 22, wherein the conditions effective to form the intermediate compound include enzymatic bromination.

25. A process for preparation of a derivative product compound of Formula (I) as follows: 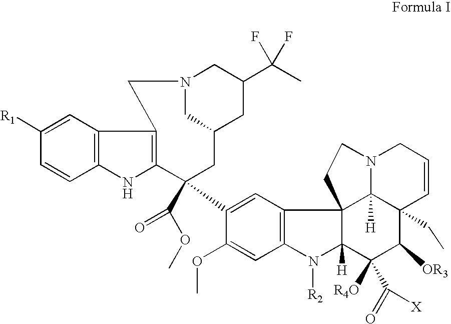
where:
R₁ is:
alkyl;
alkenyl;
alkynyl;
aryl;
heterocyclyl;
CN;
CH(O);
COR₅;
SO₂NHNH₂;
SO₂NR₅NH₂;
SO₂NR₅NHR₆;
SO₂NR₅NR₆R₇;
SO₂NHNHR₅;
SO₂NHNR₅R₆;
CO₂R₅;
SR₅;
SSR₅;
SOR₅;
SO₂R₅;
SO₂NHR₅;
SO₂NR₅R₆;
B(OR₅)₂;
CF₃;
SH;
SO₂NH₂;
NH₂;
NHR₅;
NHCOR₅;
NHSO₂R₅;
NR₅R₆;
NR₅COR₆; or
NR₅SO₂R₆;
R₅ and R₆ can form a ring;
OR₇
R₂=alkyl or CH(O);
R₃=hydrogen, alkyl, or C(O)R₅;
R₄=hydrogen or C(O)R₅;
R₅, R₆, and R₇ each are independently hydrogen, alkyl, alkenyl, alkynyl, aryl, or heterocyclyl;
X=OR₅, NR₅R₆, NHNH₂, NHNHC(O)R₅, OH, NHR₅, NH₂, or NHNHC(O)H;
R₄ and X may be linked together with intervening atoms to form a ring; or a pharmaceutically acceptable salt thereof, wherein the alkyl and alkenyl groups may be branched, straight, unsubstituted, and/or substituted and wherein the aryl, alkynyl, and heterocyclyl groups are substituted or unsubstituted, said process comprising:
converting a first intermediate compound of formula: 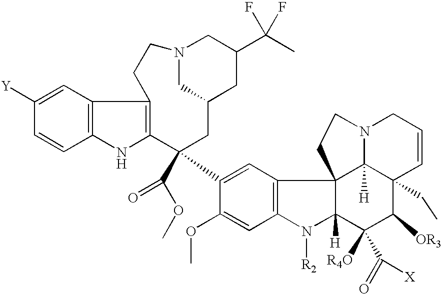
wherein Y is a halogen,
under conditions effective to produce the product compound of Formula (I).

26. The process of claim 25 further comprising:
fluorinating a second intermediate compound of the formula: 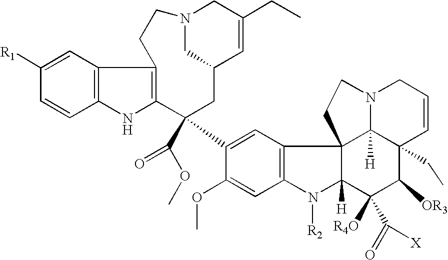
under conditions effective to produce the first intermediate compound.

27. The process of claim 26 further comprising:
converting a third intermediate compound of the formula: 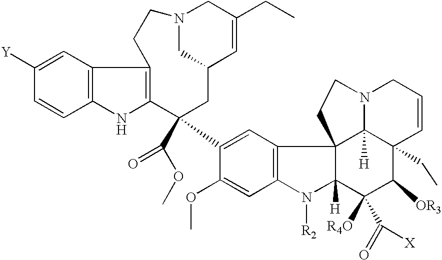
where Y is a halogen,
under conditions effective to form the second intermediate compound.

28. The process of claim 27 further comprising:
halogenating a starting material of the formula: 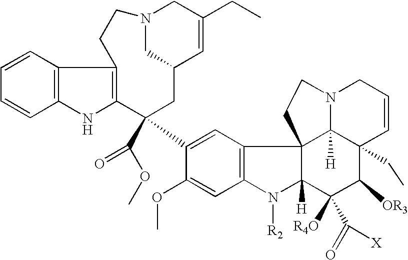
under conditions effective to produce the third intermediate compound.

29. The process of claim 28, wherein said halogenating is carried out with a halogenating agent selected from the group consisting of N-bromosuccinimide, N-iodosuccinimide, and iodine monochloride.

30. The process of claim 28, wherein the conditions effective to form the third intermediate compound include enzymatic bromination.

31. The process of claim 27, wherein said converting comprises:
reacting the third intermediate compound with a palladium catalyst reagent to produce the second intermediate compound.

32. The process of claim 31, wherein the palladium catalyst reagent is selected from the group consisting of palladium acetate, tris(dibenzylideneacetone)dipalladium(0), [1,1′-bis(diphenylphosphino)ferrocene]dichloropalladium(II), tetrakis(triphenylphosphine)palladium, and bis(triphenylphosphine) palladium(II)dichloride.

33. A composition of matter comprising the compound of claim 1 and one or more pharmaceutical excipients.

34. The composition according to claim 33, wherein R₃=acetyl.

35. The composition according to claim 33, wherein R₄=hydrogen.

36. The composition according to claim 33, wherein X=OMe.

37. The composition according to claim 33, wherein R₃=acetyl, R₄=hydrogen, and X=OMe.

38. The composition according to claim 33, wherein R₂=CH(O).

39. The composition according to claim 33, wherein R₂=alkyl.

40. A method for inhibiting cell proliferation in mammals comprising:
administering a therapeutically effective amount of the compound of claim 1 to the mammal.

41. The method according to claim 40, wherein R₃=acetyl.

42. The method according to claim 49, wherein R₄=hydrogen.

43. The method according to claim 40, wherein X=OMe.

44. The method according to claim 40, wherein R₃=acetyl, R₄=hydrogen, and X=OMe.

45. The method according to claim 40, wherein R₂=CH(O).

46. The method according to claim 40, wherein R₂=alkyl.

47. The method of claim 40, wherein the compound is administered to a mammal suffering from cancer.

48. The method of claim 47, wherein the cancer is selected from the group consisting of solid tumors, carcinomas, lymphomas, cancer diseases, Hodgkin's Disease, and neoplastic diseases.

49. The method of claim 40, wherein the mammal is human.

50. A method for treating a condition in mammals selected from the group consisting of bacterial infection, allergy, heart disease, AIDS, Human T-lymphotropic virus 1 infection, Human herpesvirus 3, Human herpesvirus 4, Human papillomavirus, diabetes mellitus, rheumatoid arthritis, Alzheimer's Disease, inflammation, arthritis, asthma, malaria, autoimmune disease, eczema, Lupus erythematosus, psoriasis, rheumatic diseases, Sjogren's syndrome, and viral infection, said method comprising
administering a therapeutically effective amount of the compound of claim 1 to the mammal.

51. The method of claim 50, wherein the mammal is human.
